(19) United States
(12) Patent Application Publication
(10) Pub. No.: US 2024/0237663 A1
(43) Pub. Date: Jul. 18, 2024

(54) METHOD OF PRODUCING FERMENTED MILK PRODUCTS WITH IMPROVED TEXTURE AND REDUCED POST-ACIDIFICATION

(71) Applicant: Chr. Hansen A/S, Hoersholm (DK)

(72) Inventors: Victoria Prebner, Hoersholm (DK); Vojislav Vojinovic, Hoersholm (DK); Maria Elina Sundberg, Hoersholm (DK)

(73) Assignee: Chr. Hansen A/S, Hoersholm (DK)

(21) Appl. No.: 18/559,720

(22) PCT Filed: May 5, 2022
(86) PCT No.: PCT/EP2022/062137

§ 371 Date: Nov. 8, 2023

(30) Foreign Application Priority Data
May 18, 2021 (EP) 21174345.5

(51) Int. Cl. A23C 9/12; A23C 9/123; C12N 1/20; C12N 9/24; C12R 1/225; C12R 1/46

(52) U.S. Cl. CPC A23C 9/1206; A23C 9/1238; C12N 1/205; C12N 9/2402; C12Y 302/01108; A23V 2400/123; A23V 2400/249; C12R 2001/225; C12R 2001/46

(57) ABSTRACT

The present invention is in the field of dairy technology. It relates to a method for producing a fermented milk product comprising the steps: (a) Providing a starter culture comprising one or more lactic acid bacteria strains selected from a group consisting of: DSM 22935, DSM 22585, DSM 22586, DSM28910 and DSM33677; (b) Adding the starter culture to a milk base to ferment said milk base until a target pH is reached; and (c) Adding a lactase to the milk base; wherein the fermented milk product has improved texture and/or reduced post-acidification as compared to a fermented milk product produced without addition of the lactase of step c) or the starter culture of step a).

### FIELD OF THE INVENTION

The present invention generally relates to methods of producing fermented milk products.

Specifically, it relates to fermented milk products with improved texture and/or reduced post-acidification and methods for producing them.

### BACKGROUND OF THE INVENTION

Fermented milk products are known in the art. Texture is an important quality parameter for fermented milk products. A smooth consistency with good mouthfeel and gel firmness is desired by many consumers.

Use of texturizing lactic acid bacteria strains for fermentation as well as addition of protein, typically skim milk powder or whey based proteins to the milk base, have been described to improve the texture of fermented milk products. Thickeners or other texturizing agents like modified starch, corn starch, pectin, gelatin, or agar are also used.

After production fermented milk products are stored for a period of time before consumption by the end-user. During storage the acidification process may continue and the decrease in pH may affect the quality of the product. Thus, such post-acidification is not desirable.

### SUMMARY OF THE INVENTION

The present invention provides a method for preparing fermented milk products. The inventors surprisingly found that it was possible to obtain improve texture and/or reduced post-acidification by using selected lactic acid bacteria strains in combination with lactase in a method for producing fermented milk products.

In a first aspect the invention relates to A method for producing a fermented milk product comprising the steps:
a) Providing a starter culture comprising one or more lactic acid bacteria strains selected from a group consisting of: DSM22935, DSM22585, DSM22586, DSM28910 and DSM33677;
b) Adding the starter culture to a milk base to ferment said milk base until a target pH is reached; and
c) Adding a lactase to the milk base;
wherein the fermented milk product has improved texture and/or reduced post-acidification as compared to a fermented milk product produced without addition of the lactase of step c) or the starter culture of step a).

In a second aspect the invention relates to A fermented milk product obtainable by the method.

In a third aspect the invention relates to A method for improving texture and/or reducing post-acidification of a fermented milk product comprising the steps:
a) Providing a starter culture comprising one or more lactic acid bacteria strains selected from a group consisting of: DSM22935, DSM22585, DSM22586, DSM28910 and DSM33677;
b) Adding the starter culture to a milk base to ferment said milk base until a target pH is reached; and
c) Adding a lactase to the milk base.

In a fourth aspect the invention relates to A composition comprising a lactase and two lactic acid bacteria strains selected from a group consisting of: DSM22935+DSM22586; DSM22935+DSM33677; DSM22935+DSM28910; DSM22935+DSM22585; DSM22586+DSM33677; DSM22586+DSM28910; DSM22586+DSM22585; DSM33677+DSM28910; DSM33677+DSM22585; or DSM28910+DSM22585.

### Definitions

The expression “lactic acid bacteria” (“LAB”) designates gram-positive, microaerophilic or anaerobic bacteria, which ferment sugars with the production of acids including lactic acid as the predominantly produced acid, acetic acid and propionic acid. The industrially most useful lactic acid bacteria are found within the order “Lactobacillales” which includes Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. These are frequently used as food cultures alone or in combination with other lactic acid bacteria.

Lactic acid bacteria, including bacteria of the species Lactobacillus sp. and Lactococcus sp., are normally supplied to the dairy industry either as frozen or freeze-dried cultures for bulk starter propagation or as so-called “Direct Vat Set” (DVS™) cultures, intended for direct inoculation into a fermentation vessel or vat for the production of a dairy product, such as a fermented milk product or a cheese. Such lactic acid bacterial cultures are in general referred to as “starter cultures” or “starters”. Typically, a starter culture for yogurt comprises Streptococcus thermophilus and Lactobacillus delbrueckii subsp. bulgaricus, and in most countries a yogurt is by legislation defined as a fermented milk product produced using a starter culture comprising the two said strains.

The term “fermentation” means the conversion of carbohydrates into alcohols or acids through the action of a microorganism. Preferably, fermentation in the methods of the invention comprises conversion of lactose to lactic acid. Fermentation processes to be used in production of dairy products are well known and the person of skill in the art will know how to select suitable process conditions, such as temperature, oxygen, amount and characteristics of microorganism(s) and process time. Obviously, fermentation conditions are selected so as to support the achievement of the present invention, i.e. to obtain a dairy product in solid (such as a cheese) or liquid form (such as a fermented milk product).

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DETAILED DESCRIPTION OF THE INVENTION

This invention relates to fermented milk products and methods of producing said products.

### Method of Producing a Fermented Milk Product

In one embodiment the invention relates to A method for producing a fermented milk product comprising the steps:
a) Providing a starter culture comprising one or more lactic acid bacteria strains selected from a group consisting of: DSM22935, DSM22585, DSM22586, DSM28910 and DSM33677;
b) Adding the starter culture to a milk base to ferment said milk base until a target pH is reached; and
c) Adding a lactase to the milk base;
wherein the fermented milk product has improved texture and/or reduced post-acidification as compared to a fermented milk product produced without addition of the lactase of step c) or the starter culture of step a).

In one embodiment the invention relates to The method, wherein the starter culture comprises DSM22935, and one or more lactic acid bacteria strains selected from a group consisting of DSM22585, DSM22586, DSM28910, and DSM33677.

In one embodiment the invention relates to The method, wherein the starter culture comprises DSM22935+DSM22586; DSM22935+DSM33677; DSM22935+DSM28910; DSM22935+DSM22585; DSM22586+DSM33677; DSM22586+DSM28910; DSM22586+DSM22585; DSM33677+DSM28910; DSM33677+DSM22585; or DSM28910+DSM22585.

In one embodiment the invention relates to The method, wherein the starter culture comprises DSM22935+DSM22586+DSM33677; DSM22935+DSM22586+DSM28910; DSM22935+DSM22586+DSM22585; DSM22935+DSM33677+DSM28910; DSM22935+DSM33677+DSM22585; or DSM22935+DSM28910+DSM22585.

In one embodiment the invention relates to The method, wherein the lactic acid bacteria strains are added to the milk base in a concentration of 1.0 E04 to 1.0 E12 CFU/g, 1.0 E04 to 1.0 E11, 1.0 E04 to 1.0 E10, 1.0 E04 to 1.0 E09, 1.0 E05 to 1.0 E08, 1.0 E05 to 1.0 E07, 1.0 E05 to 1.0 E06, or 1.0E04, 1.0E05, 1.0E06, 1.0E07, 1.0E08, 1.0E09, 1.0E10, 1.0E11, or 1.0E12.

In connection with strains of the genus Lactobacillus, the term “CFU” means colony forming units as determined by growth (forming a colony) on an MRS agar plate incubated at anaerobic conditions at 37° C. for 3 days. The MRS agar has the following composition (g/l): Bacto Proteose Peptone No. 3: 10.0
Bacto Beef extract: 10.0
Bacto Yeast extract: 5.0
Dextrose: 20.0
Sorbitan Monooleate Complex: 1.0
Ammonium Citrate: 2.0
Sodium Acetate: 5.0
Magnesium Sulfate: 0.1
Manganese Sulfate: 0.05
Potassium Phosphate Dibasis: 2.0
Bacto Agar: 15.0
Milli-Q water: 1000 ml.

pH is adjusted to 5.4 or 6.5: pH is adjusted to 6.5 for L. rhamnosus, L. casei and L. paracasei. For all other Lactobacillus species the pH is adjusted to 5.4. In particular, pH is adjusted to 5.4 for L. delbrueckii subsp. bulgaricus; L. acidophilus and L. helveticus. pH is adjusted to 6.5 for L. rhamnosus, L. casei and L. paracasei.

In connection with S. thermophilus, the term “CFU” means colony forming units as determined by growth (forming a colony) on an M 17 agar plate incubated at aerobic conditions at 37° C. for 3 days. The M 17 agar has the following composition (g/l):
Tryptone: 2.5 g
Peptic digest of meat: 2.5 g
Papaic digest of soybean meal: 5.0 g
Yeast extract: 2.5 g
Meat extract: 5.0 g
Lactose: 5.0 g
Sodium-glycero-phosphate: 19.0 g
Magnesium sulphate, 7 H₂O: 0.25 g
Ascorbic acid: 0.5 g
Agar: 15.0 g
Milli-Q water: 1000 ml.

pH is adjusted to final pH 7.1±0.2 (25° C.) In one embodiment the invention relates to The method according to any one of the preceding claims, wherein the target pH is in the range of 3.0-6.0; 3.5-5.5; 4.0-5.0; preferably 4.00; 4.10; 4.20; 4.30; 4.40; 4.45; 4.50; 4.55; 4.60; 4.65; 4.70; 4.80; 4.90; or 5.00.

The term “target pH” means the pH at which the fermentation step ends. Depending on various parameters of the process, the fermentation step is terminated by a method selected from the group consisting of 1) acidification of the fermented milk rendering at least one strain of the starter culture unable to grow, 2) cooling treatment; and 3) depletion of fermentation substrate.

It was surprisingly found that the method of the invention wherein a starter culture was used in combination with a lactase resulted in a fermented milk product with improved texture. Improved texture may be expressed by e.g. viscosity, ropiness, and/or gel firmness.

Viscosity (unit is Pas) is defined as “shear stress” (Pa)/shear rate (1/s). Rheological properties of non-newtonian liquid, such as fermented milks, can be presented in form of a so-called flow curve, which depicts measured shear stress values for a range of shear rates. It can be shown that the shear stress measured at the shear rate of 60/s correlates well with the viscosity perceived by the sensory panel when the yoghurt is taken into the mouth and moved by the taster's tongue. Similarly, the shear stress measured at 300/s correlates well with the resistance to flow sensed in the back of the mouth and the throat upon swallowing.

In connection with the present invention, “shear stress” may be measured by the following method: Seven days after production, the fermented milk product was brought to 13° C. and mixed well by means of a spoon in 5 gentle circular moves to ensure homogeneity.

The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:
1. Five minutes holding time without any physical stress.
2. Oscillation step (to measure the elastic and viscous modulus, G′ and G″, respectively, therefore calculating the complex modulus G*). Constant strain=0.3%, frequency (f)=[0.5 . . . 8] Hz. 6 measuring points over 60s (one every 10s).
3. Rotation step: shear stress is measured while shear rate is progressively increased from 0.3 to 300/s, followed by progressive decrease from 300 to 0.3/s. Each step contained 21 measuring points over 210 s (on every 10 s).

In connection with the present invention, “gel firmness” may be measured by the following method: Complex Modulus G* was evaluated by oscillation measurement using ASC rheometer model DSR502 from Anton Paar. The method is based on an oscillation step, where the sample is oscillated between two surfaces, with the upper geometry (bob) moving and the lower cup remaining stationary. The oscillation is performed from 0.5-8 Hz at constant strain. For these evaluations the results are extracted from measurements at 1.52 Hz. Samples are placed at 13° C. for 1 hour prior to measuring. Each sample is gently stirred with a spoon 5 times from bottom to top to assure a homogenous sample. The rheology cups are filled until the line and placed in the sample magazine. Samples are measured in duplicates using two separate yogurt cups. Measurements are conducted at day +7 and day +28 and temperature of measurement is set to 13° C. Samples are stored at 6° C. until the day of measurement.

In one embodiment the invention relates to The method according to any one of the preceding claims, wherein texture measured at 13° C. as gel firmness by complex modulus in Pa is increased with 5-10%; 10-15%; 15-20%; 20-25%; 25-30%; 30-35%; 35-40%; 40-45%; 45-50%; or at least 5%; 10%; 15%; 20%; 25%; 30%; 35%; 40%; 45%; or at least 50% when stored from termination of fermentation for 7 or 28 days at a temperature of 6° C.

It was surprisingly found that the method of the invention wherein a starter culture was used in combination with a lactase resulted in a fermented milk product with a reduced post-acidification. In one embodiment the invention relates to The method, wherein pH of the fermented milk product is maintained within a range of 0.30; 0.25; 0.20; 0.15; 0.10; or 0.05 pH units when stored from termination of fermentation for 7 or 28 days at a temperature of 6° C. or 25° C.

In a further embodiment the present invention relates to A method for improving texture and/or reducing post-acidification of a fermented milk product comprising the steps:
a) Providing a starter culture comprising one or more lactic acid bacteria strains selected from a group consisting of: DSM 22935, DSM 22585, DSM 22586, DSM28910 and DSM33677;
b) Adding the starter culture to a milk base to ferment said milk base until a target pH is reached; and
c) Adding a lactase to the milk base.

### Milk Base

The fermented milk product is made by fermenting a milk base.

The term “milk” is to be understood as the lacteal secretion obtained by milking of any mammal, such as cows, sheep, goats, buffaloes or camels. In a preferred embodiment, the milk is cow's milk. The term milk includes any solution comprising lactose. Such solutions may also be protein/fat solutions comprising plant materials, e.g. soy milk.

The term “milk base” may be any raw and/or processed milk material that can be subjected to fermentation according to the method of the invention. Thus, useful milk bases include, but are not limited to, solutions/-suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk base may originate from any mammal, e.g. being substantially pure mammalian milk, or reconstituted milk powder.

The protein content of such milk base may vary depending of which fermented milk product is desired. In one embodiment the invention relates to The method, wherein the milk base has a protein level in the range of 1-10%; 2-8%; 2-6%; 3-5%; such as 3.0%; 3.5%; 4.0%; 4.5%; 5.0%; 5.5%; or 6.0% by weight.

Prior to fermentation, the milk base may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk base to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

### Starter Culture & Composition

To ferment a milk base a starter culture is added. The term “starter” or “starter culture” as used in the present context refers to a composition or culture comprising one or more food-grade microorganisms in particular lactic acid bacteria (LAB), which are responsible for the fermentation and acidification of the milk base. The starter culture may be a yogurt starter culture. Starter cultures may be fresh, frozen or freeze-dried. It is within the skills of ordinary practitioners to determine the starter culture and amounts to be used.

The microorganisms may be thermophilic or mesophilic depending on the desired fermented milk product.

The term “thermophile” herein refers to microorganisms that thrive best at temperatures above 35° C. The industrially most useful thermophilic bacteria include Streptococcus spp. and Lactobacillus spp. The term “thermophilic fermentation” herein refers to fermentation at a temperature above about 35° C., such as between 35° C. to 45° C. The term “thermophilic fermented milk product” refers to fermented milk products prepared by thermophilic fermentation of a thermophilic starter culture and include such fermented milk products as set-yoghurt, stirred-yoghurt and drinking yoghurt, e.g. Yakult.

The term “mesophile” herein refers to microorganisms that thrive best at moderate temperatures (15° C.-35° C.). The industrially most useful mesophilic bacteria include Lactococcus spp. and Leuconostoc spp. The term “mesophilic fermentation” herein refers to fermentation at a temperature between 22° C. to 35° C. The term “mesophilic fermented milk product” refers to fermented milk products prepared by mesophilic fermentation of a mesophilic starter culture and include such fermented milk products as buttermilk, sour milk, cultured milk, smetana, sour cream, Kefir and fresh cheese, such as quark, tvarog and cream cheese.

In one embodiment the invention relates to a starter culture comprising one, two, three, four, or five lactic acid bacteria strains. In one embodiment the invention relates to a starter culture comprising one lactic acid bacteria selected from DSM22935; DSM22586; DSM33677; DSM28910; or DSM22585. In one embodiment the invention relates to a starter culture comprising two lactic acid bacteria selected from DSM22935+DSM22586; DSM22935+DSM33677; DSM22935+DSM28910; DSM22935+DSM22585; DSM22586+DSM33677; DSM22586+DSM28910; DSM22586+DSM22585; DSM33677+DSM28910; DSM33677+DSM22585; or DSM28910+DSM22585. In one embodiment the invention relates to a starter culture comprising three lactic acid bacteria selected from DSM22935+DSM22586+DSM33677; DSM22935+DSM22586+DSM28910; DSM22935+DSM22586+DSM22585; DSM22935+DSM33677+DSM28910; DSM22935+DSM33677+DSM22585; or DSM22935+DSM28910+DSM22585. Preferably the starter culture comprises three lactic acid bacteria selected from DSM22935+DSM22586+DSM33677 or DSM22935+DSM28910+DSM22585. In one embodiment the invention relates to a starter culture comprising four lactic acid bacteria selected from DSM22935+DSM22586+DSM33677+DSM28910; DSM22935+DSM22586+DSM33677+DSM22585; DSM22935+DSM22586+DSM28910+DSM22585; or DSM22935+DSM22586+DSM22585+DSM33677. In one embodiment the invention relates to a starter culture comprising five lactic acid bacteria selected from DSM22935+DSM22586+DSM33677+DSM28910+DSM22585.

In one embodiment the invention relates to a starter culture comprising DSM22935 and one or more lactic acid bacteria selected from the group consisting of DSM22586; DSM33677; DSM28910; and DSM22585. In one embodiment the invention relates to a starter culture comprising DSM22586 and one or more lactic acid bacteria selected from the group consisting of DSM22935; DSM33677; DSM28910; and DSM22585. In one embodiment the invention relates to a starter culture comprising DSM33677 and one or more lactic acid bacteria selected from the group consisting of DSM22935; DSM22586; DSM28910; and DSM22585. In one embodiment the invention relates to a starter culture comprising DSM28910 and one or more lactic acid bacteria selected from the group consisting of DSM22935; DSM22586; DSM33677; and DSM22585. In one embodiment the invention relates to a starter culture comprising DSM22585 and one or more lactic acid bacteria selected from the group consisting of DSM22935; DSM22586; DSM33677; and DSM28910.

The starter culture may further comprise a lactase. The lactase may be a lactase as described in the section “Lactase” below.

In one embodiment the invention relates to a starter culture comprising a lactase and one or more lactic acid bacteria selected from a group consisting of: DSM22935; DSM22586; DSM33677; DSM28910; and DSM22585. The specific combination of lactic acid bacteria strains may be any of the combinations described supra.

In one embodiment the invention relates to a starter culture comprising a lactase and one, two, three, four, or five lactic acid bacteria strains. In one embodiment the invention relates to a starter culture comprising a lactase and one lactic acid bacteria selected from DSM22935; DSM22586; DSM33677; DSM28910; or DSM22585. In one embodiment the invention relates to a starter culture comprising a lactase and two lactic acid bacteria selected from DSM22935+DSM22586; DSM22935+DSM33677; DSM22935+DSM28910; DSM22935+DSM22585; DSM22586+DSM33677; DSM22586+DSM28910; DSM22586+DSM22585; DSM33677+DSM28910; DSM33677+DSM22585; or DSM28910+DSM22585. In one embodiment the invention relates to a starter culture comprising a lactase and three lactic acid bacteria selected from DSM22935+DSM22586+DSM33677; DSM22935+DSM22586+DSM28910; DSM22935+DSM22586+DSM22585; DSM22935+DSM33677+DSM28910; DSM22935+DSM33677+DSM22585; or DSM22935+DSM28910+DSM22585. Preferably the starter culture comprises a lactase and three lactic acid bacteria selected from DSM22935+DSM22586+DSM33677 or DSM22935+DSM28910+DSM22585. In one embodiment the invention relates to a starter culture comprising a lactase and four lactic acid bacteria selected from DSM22935+DSM22586+DSM33677+DSM28910; DSM22935+DSM22586+DSM33677+DSM22585; DSM22935+DSM22586+DSM28910+DSM22585; or DSM22935+DSM22586+DSM22585+DSM33677. In one embodiment the invention relates to a starter culture comprising a lactase and five lactic acid bacteria selected from DSM22935+DSM22586+DSM33677+DSM28910+DSM22585.

The starter culture of the present invention may be provided in several forms. It may be a frozen form, dried form, freeze dried form, or liquid form. Thus, in one embodiment the starter culture is in frozen, dried, freeze-dried or liquid form.

The starter culture of the present invention may additionally comprise cryoprotectants, lyoprotectants, antioxidants, nutrients, fillers, flavorants or mixtures thereof. The starter culture preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectantare known to a skilled person in the art. Suitable cryoprotectants or lyoprotectants include mono-, di-, tri and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex mixtures (such as skim milk, peptones, gelatin, yeast extract) and inorganic compounds (such as sodium tripolyphosphate). In one embodiment, the starter culture according to the present invention may comprise one or more cryoprotective agent(s) selected from the group consisting of inosine-5′-monophosphate (IMP), adenosine-5′-monophosphate (AMP), guanosine-5′-monophosphate (GMP), uranosine-5′-monophosphate (UMP), cytidine-5′-monophosphate (CMP), adenine, guanine, uracil, cytosine, adenosine, guanosine, uridine, cytidine, hypoxanthine, xanthine, hypoxanthine, orotidine, thymidine, inosine and a derivative of any such compounds. Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The starter culture may optionally comprise further substances including fillers (such as lactose, maltodextrin) and/or flavorants.

In one embodiment of the invention the cryoprotective agent is an agent or mixture of agents, which in addition to its cryoprotectivity has a booster effect. The expression “booster effect” is used to describe the situation wherein the cryoprotective agent confers an increased metabolic activity (booster effect) on to the thawed or reconstituted culture when it is inoculated into the medium to be fermented or converted. Viability and metabolic activity are not synonymous concepts. Commercial frozen or freeze-dried cultures may retain their viability, although they may have lost a significant portion of their metabolic activity e.g. cultures may lose their acid-producing (acidification) activity when kept stored even for shorter periods of time. Thus, viability and booster effect has to be evaluated by different assays. Whereas viability is assessed by viability assays such as the determination of colony forming units, booster effect is assessed by quantifying the relevant metabolic activity of the thawed or reconstituted culture relative to the viability of the culture. The term “metabolic activity” refers to the oxygen removal activity of the cultures, its acid-producing activity, i. e. the production of e. g. lactic acid, acetic acid, formic acid and/or propionic acid, or its metabolite producing activity such as the production of aroma compounds such as acetaldehyde, (a-acetolactate, acetoin, diacetyl and 2,3-butylene glycol (butanediol)).

In one embodiment the starter culture of the invention contains or comprises from 0.2% to 20% of the cryoprotective agent or mixture of agents measured as % w/w of the material. It is, however, preferable to add the cryoprotective agent or mixture of agents at an amount which is in the range from 0.2% to 15%, from 0.2% to 10%, from 0.5% to 7%, and from 1% to 6% by weight, including within the range from 2% to 5% of the cryoprotective agent or mixture of agents measured as % w/w of the frozen material by weight. In a preferred embodiment the culture comprises approximately 3% of the cryoprotective agent or mixture of agents measured as % w/w of the material by weight. The amount of approximately 3% of the cryoprotective agent corresponds to concentrations in the 100 mM range. It should be recognized that for each aspect of embodiment of the invention the ranges may be increments of the described ranges.

In a further aspect, the starter culture of the present invention contains or comprises an ammonium salt (e.g. an ammonium salt of organic acid (such as ammonium formate and ammonium citrate) or an ammonium salt of an inorganic acid) as a booster (e.g. growth booster or acidification booster) for bacterial cells, such as cells belonging to the species S. thermophilus, e.g. (substantial) urease negative bacterial cells. The term “ammonium salt”, “ammonium formate”, etc, should be understood as a source of the salt or a combination of the ions. The term “source” of e.g. “ammonium formate” or “ammonium salt” refers to a compound or mix of compounds that when added to a culture of cells, provides ammonium formate or an ammonium salt. In some embodiments, the source of ammonium releases ammonium into a growth medium, while in other embodiments, the ammonium source is metabolized to produce ammonium. In some preferred embodiments, the ammonium source is exogenous. In some particularly preferred embodiments, ammonium is not provided by the dairy substrate. It should of course be understood that ammonia may be added instead of ammonium salt. Thus, the term ammonium salt comprises ammonia (NH3), NH40H, NH4+, and the like.

In one embodiment the starter culture of the invention may comprise thickener and/or stabilizer, such as pectin (e.g. HM pectin, LM pectin), gelatin, CMC, Soya Bean Fiber/Soya Bean Polymer, starch, modified starch, carrageenan, alginate, and guar gum.

In one embodiment wherein the microorganism produces a polysaccharide (such as EPS) which causes a high/ropy texture in the acidified milk product the acidified milk product is produced substantially free, or completely free of any addition of thickener and/or stabilizer, such as pectin (e.g. HM pectin, LM pectin), gelatin, CMC, Soya Bean Fiber/Soya Bean Polymer, starch, modified starch, carrageenan, alginate, and guar gum. By substantially free should be understood that the product comprises from 0% to 20% (w/w) (e.g. from 0% to 10%, from 0% to 5% or from 0% to 2% or from 0% to 1%) thickener and/or stabilizer.

The starter culture and compositions describe above may be provided as a mixture or as a kit-of-parts comprising a lactase and a starter culture comprising one or more lactic acid bacteria. The lactic acid bacteria may be any lactic acid bacteria or any combination of lactic acid bacteria as describes above.

### Lactase

A lactase in the context of the present invention is a glycoside hydrolase having the ability to hydrolyze the disaccharide lactose into constituent galactose and glucose monomers.

The group of lactases, to which the lactase of the invention belongs, comprises but is not limited to enzymes assigned to subclass EC 3.2.1.108. Enzymes assigned to other subclasses, such as, e.g., EC 3.2.1.23, may also be lactases in the context of the present invention. A lactase in the context of the invention may have other activities than the lactose hydrolyzing activity, such as for example a transgalactosylating activity. In the context of the invention, the lactose hydrolyzing activity of the lactase may be referred to as its lactase activity or its beta-galactosidase activity.

Enzymes having lactase activity to be used in a method of the present invention may be of animal, of plant or of microbial origin. Preferred lactases are obtained from microbial sources, in particular from a filamentous fungus or yeast, or from a bacterium.

The enzyme may, e.g., be derived from a strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Kluveromyces, e.g. K. fragilis, K. lactis; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Sclerotinia, e.g. S. libertiana; Torula; Torulopsis; Trichophyton, e.g. T. rubrum; Whetzelinia, e.g. W. sclerotiorum; Bacillus, e.g. B. coagulans, B. circulans, B. megaterium, B. novalis, B. subtilis, B. pumilus, B. stearothermophilus, B. thuringiensis; Bifidobacterium, e.g. B. longum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica.

In a preferred embodiment, the lactase originates from a bacterium, e.g. from the family Bifidobacteriaceae, such as from the genus Bifidobacterium, such as from a strain of B. bifidum, B. animalis or B. longum. In a more preferred embodiment, the lactase originates from Bifidobacterium bifidum.

In another preferred embodiment, the lactase originates from a fungus, e.g. from the family Saccharomycetaceae, such as from the genus Kluyveromyces, such as from a strain of K. fragilis or K. lactis. In a more preferred embodiment, the lactase originates from K. lactis.

In one embodiment the invention relates to The method, wherein the lactase is added at the start, during, or at the end of the fermentation in step b).

The term “at the start of the fermentation step” means shortly before, at the same time as or shortly after addition of the starter culture to the milk base. Here, the term “shortly” means less than 30 minutes”. The term “during the fermentation step” means at any time during the fermentation after the start and before the end of the fermentation. The term “at the end of the fermentation step” means shortly before, at the same time as or shortly after the target pH is reached. Here, the term “shortly” means less than 30 minutes”.

In one embodiment the invention relates to The method, wherein the lactase is selected from a group consisting of a neutral lactase; an acidic lactase; and a low pH stable lactase.

The term “neutral lactase” means a lactase with a pH optimum around neutral pH typically in the pH range of 6.0 to 8.0. Examples of neutral lactases are Ha-lactase™ (Chr. Hansen A/S), Bonlacta (Dupont), Godo-YNL2 (Dupont), Maxilact® (DSM).

The term “acidic lactase” means a lactase with a pH optimum at an acidic pH typically in the pH range of 3.5 to 5.5. Examples of acidic lactates are Maxilact® A4 and those disclosed in WO2020/079116.

The term “low pH stable lactase” herein refers to a lactase, which retains its activity at a pH of 5.0 and a temperature of 37° C. at a level of at least 5% as compared to its activity at the optimum pH of the lactase. The term “activity at the optimum pH” means the lactase activity at the pH, where the lactase has its optimum activity. Examples of low pH stable lactases are NOLA®Fit (Chr. Hansen A/S), Saphera® (Novozymes A/S).

Other suitable lactases are Nola Fiber (Chr. Hansen A/S), Saphera Fiber (Novozyme A/S), Nurica (Dupont).

In one embodiment the invention relates to The method, wherein lactase is added to the milk base in a volumetric activity of 500 to 50.000 NLU/L; 1000-40.000; 1500-30.000; 2000-20.000; 3000-10.000; 4000-9000; 5000-8000; or 6000-7000 NLU/L.

In one embodiment the invention relates to The method, wherein lactase is added to the milk base in a volumetric activity of 500 to 50.000 BLU/L; 1000-40.000; 1500-30.000; 2000-20.000; 3000-10.000; 4000-9000; 5000-8000; or 6000-7000 BLU/L.3

The activity of a specific lactase may be determined by direct measurement of glucose released from lactose. The skilled person will know how to determine such activity. Alternatively, the activity may be determined by using the lactase activity assay described below or in Example 1 of the present application.

Lactase activity for a neutral lactase such as Ha-Lactase was determined as given below. Lactase activity was determined as Neutral Lactase Units (NLU) using o-nitrophenyl-β-D-galactopyranoside (ONPG) as the substrate, according to the procedure described in FCC (fourth ed, July 1996, p801-802: Lactase (neutral) β-galactosidase activity).

Lactase activity of a low pH stable lactase such as NOLA® Fit product was based on the hydrolysis of ONPG (o-nitrophenyl β-D-galactopyranoside) into β-D-galactose and ONP (o-nitrophenol). The ONP has yellow color and can be quantified by measurement of absorbance at 405 nm using a spectrophotometer. The NOLA® Fit activity is determined relative to a lactase enzyme standard and the activity unit is defined as Bifido Lactase Unit per gram of the product and abbreviated as BLU/g. Lactase sample of known BLU activity can be obtained from Chr. Hansen A/S, Denmark.

Both the standard and test enzymes were diluted in MES buffer pH 6.5 (50 mM, containing 1 mM MgSO₄ and 0.045% Brij). The substrate ONPG (1.46 mg/ml) was dissolved in the MES buffer (as described above). The reaction was started by mixing of pre-heated 0.5 ml of standard or test sample (30° C.) with 3.5 ml preheated substrate solution. The reaction mixture was incubated for 10 min at 30° C. After 10 min incubation, the reaction was stopped by adding 1 ml of stop solution (1 M Na₂CO₃). The absorbance of the solution was measured at 405 nm.

In the current context, 1 BLU is defined as the amount of enzyme which releases 1 micromole glucose per minute in M-buffer at pH 6.5 at 37C with a lactose concentration of 4.75% w/v. M-buffer is prepared by dissolving 3.98 g C₆H₅Na₃O₇-2H₂O, 8.31 g citric acid, 0.9 g K₂SO₄, 2.6 g K₂HPO₄, 7.35 g KH₂PO₄, 5.45 g KOH, 4.15 g MgCI₂-6H₂O, 3.75 g CaCI₂-2H₂O and 1.4 g NaHCO₃ in 4 liter water, adding 12.5 ml 4N NaOH, adjusting to pH 6.5 using HCl, and adding water up to a total volume of 5 liter.

### Fermented Milk Product

The present invention furthermore relates to A fermented milk product obtainable by the method of the invention.

In one embodiment the invention relates to A fermented milk product with improved texture and/or reduced post-acidification, wherein the product comprises a lactase and a starter culture comprising one or more lactic acid bacteria strains selected from a group consisting of: DSM 22935, DSM 22585, DSM 22586, DSM28910 and DSM33677.

The term “fermented milk product” means a product wherein the preparation of the product involves fermentation of a milk base with lactic acid bacteria or a starter culture. “Fermented milk product” as used herein includes but is not limited to products such as thermophilic fermented milk products, e.g. yoghurt, mesophilic fermented milk products, e.g. sour cream and buttermilk, as well as fermented whey.

In one embodiment of the invention, the fermented milk product is a product comprising a lactic acid bacteria strain selected from Streptococcus thermophilus and/or Lactobacillus delbrueckii subsp. bulgaricus.

In one embodiment the fermented milk product comprises one or more lactic acid bacteria selected from the group consisting of DSM22935; DSM22586; DSM33677; DSM28910; and DSM22585. The fermented milk product may comprise any combination of the strains DSM22935; DSM22586; DSM33677; DSM28910; and DSM22585 as described in detail in the paragraph “Starter Culture & Composition” supra.

In one embodiment of the invention, the fermented milk product is selected form the group consisting of yogurt, fresh cheese, cream cheese, tvarog, quark, sour milk, sour cream, buttermilk, crème fraiche, fromage frais, skyr, fermented whey, cultured milk, smetana, kefir, drinking yogurt, and Yakult. Preferably, the yogurt is selected from set yogurt, stirred yogurt and drinking yogurt. Preferably the cheese is selected from quark, tvarog and cream cheese.

In one embodiment of the invention, the fermented milk product comprises a further food product selected from the group consisting of fruit beverage, fermented or unfermented cereal products, chemically acidified or unacidified cereal products, soy milk products and any mixture thereof. The fermented milk product of the invention may also comprise vanilla, coffee, sucrose, or artificial sweeteners.

The fermented milk product typically contains protein in an amount of between 2.0% to 3.5% by weight. The fermented milk product may also be a low protein product with a protein level of between 1.0% to 2.0% by weight. Alternatively, the fermented milk product may be a high protein product with a protein level above 3.5% by weight.

In a further embodiment the present invention relates to Use of a lactase and a starter culture for improving texture and/or reducing post-acidification of a fermented milk product, wherein the lactase is selected from a group consisting of a neutral lactase; an acidic lactase; and a low pH stable lactase. In one embodiment the present invention relates to a Use, wherein the starter culture comprises one or more lactic acid bacteria strains selected from a group consisting of: DSM22935, DSM22585, DSM22586, DSM28910 and DSM33677. Specific combinations of suitable strains are described in the section “Starter Culture & Composition”.

### Taxonomy

It will be appreciated that the Lactobacillus genus taxonomy was updated in 2020. The new taxonomy is disclosed in Zheng et al. 2020 (Zheng et al., Int. J. Syst.Evol.Microbiol. DOI 10.1099/ijsem.0.004107) and will be cohered to herein if not otherwise indicated. For the purpose of the present invention, the table below presents a list of new and old names of some Lactobacillus species relevant to the present invention.

TABLE 1
New and old names of some relevant Lactobacillus species.

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei subsp. paracasei | Lacticaseibacillus paracasei subsp. Paracasei |
| Lactobacillus plantarum subsp. plantarum | Lactiplantibacillus plantarum subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

### Deposit and Expert Solution

The applicant requests that a sample of the deposited microorganisms stated in the table below may only be made available to an expert, subject to available provisions governed by Industrial Property Offices of States Party to the Budapest Treaty, until the date on which the patent is granted.

TABLE 2
Deposits made at a Depositary institution having acquired the status
of international depositary authority under the Budapest Treaty on the
International Recognition of the Deposit of Microorganisms for the
Purposes of Patent Procedure: Leibniz Institute DSMZ-German
Collection of Microorganisms and Cell Cultures Inhoffenstr. 7B,
38124 Braunschweig, Germany.

| Species of the Deposited Strain | Accession No. | Deposit date |
|---|---|---|
| Streptococcus thermophilus | DSM 33677 | 2020 Oct. 29 |

The following strains have been deposited and were described in the indicated published patent applications. The Streptococcus thermophilus strains: DSM22935 (WO2011/092300); DSM22586 (WO2011/000879); DSM22585 (WO2010/023178). The Lactobacillus delbrueckii subsp. bulgaricus strain: DSM28910 (WO2015/193459).

### EXAMPLES

### Material and Methods

### Milk Base:

Different milk compositions were prepared by blending pasteurized milk, skimmed milk powder and water in appropriate ratios to reach the desired final composition by weight.

TABLE 3
Composition of Milk Base

| Milk Base | Ingredients | Protein (%) | Fat (%) |
|---|---|---|---|
| MB1 | Fresh milk 1.5% fat (Arla), Fresh | 3.50% | 1.50% |
| | milk 3.5% milk (Arla), water | | |
| MB2 | Fresh milk 1.5% fat (Arla), Skim | 4.00% | 1.50% |
| | Milk Powder (Arla), water | | |
| MB3 | Fresh milk 1.5% fat (Arla), Skim | 4.50% | 1.50% |
| | Milk Powder (Arla), water | | |

### Starter Culture and Lactase:

Culture 1 comprises the strains DSM 22586, DSM 22935, and DSM 33677.

Culture 2 comprises the strains DSM 22585, DSM 22935, and DSM 28910.

Culture 3 comprises the strains DSM 22586 and DSM 22935.

Culture 4 comprises the strain DSM 22935.

Low pH stable lactase: NOLA™ Fit 5500\6X1L (Chr. Hansen A/S)

Neutral lactase: Ha-Lactase 5200\6X1Kg (Chr. Hansen A/S)

### Fermentation Conditions and Post Treatment:

The inoculated milk samples were incubated at the set temperature by use of water bath, and the pH was continuously monitored. As soon as a target pH of 4.55 was reached the sample was removed from the water bath, smoothened and cooled to the storage temperature of 6° C.

TABLE 4
Fermentation

| Fermentation | |
|---|---|
| scale | 3 L/5 L |
| Preparation | Disperse powder in cold milk (<10° C.); |
| of milk base | Heat to 58° C. ± 2° C.; |
| | Re-hydrate for 1 hour at 58° C. ± 2° C. with stirring; |
| | Homogenization at 200/50 bar and 65° C.; |
| | Pasteurization at 95° C. for 5 min. |
| Culture +/− | Warm inoculation at 43° C. with |
| Enzyme | 500 U/2500 L Lactic acid bacteria; |
| inoculation | 4000 BLU/L NOLA ™ Fit |
| | 7500 NLU/L Ha-Lactase |
| Fermentation | 43° C. |
| temperature | |
| Target pH | pH 4.55 measured with pH meter calibrated at 43° C. |
| Post treatment | Apply 2 bar before heat exchanger with |
| | outlet temperature set to 20° C. |

### Assays:

Post-acidification was measured with a pH meter following pH over a period of 7 or 28 days in samples stored at 6° C.

Gel firmness was measured by Complex Modulus G* which was evaluated by oscillation measurement using ASC rheometer model DSR502 from Anton Paar. The method is based on an oscillation step, where the sample is oscillated between two surfaces, with the upper geometry (bob) moving and the lower cup remaining stationary. The oscillation is performed from 0.5-8 Hz at constant strain. For these evaluations the results are extracted from measurements at 1.52 Hz. Samples are placed at 13° C. for 1 hour prior to measuring. Each sample is gently stirred with a spoon 5 times from bottom to top to assure a homogenous sample. The rheology cups are filled until the line and placed in the sample magazine. Samples are measured in duplicates using two separate yogurt cups.

Measurements are conducted at day +7 and day +28 and temperature of measurement is set to 13° C. Samples are stored at 6° C. until the day of measurement.

In order to assess the impact the lactases on started cultures for textural properties and post acidification overshelf life, several yogurt production were carried out where started cultures, lactases and milk base were combined as described in the table below. The results of the fermentation in terms gel firmness and post acidification are shown in Example 1 and 2.

TABLE 5
Trial set up.

| | | | | Culture |
|---|---|---|---|---|
| Trial | Culture | Milk base | Lactase | Inoculation |
| 1 | Culture 1 | MB 2 (4%) | NA | 200 U/1000 L |
| 2 | Culture 1 | MB 2 (4%) | NOLA ™ Fit | 200 U/1000 L |
| | | | (4000 BLU/L) | |
| 3 | Culture 1 | MB 2 (4%) | Ha-lactase | 200 U/1000 L |
| | | | (7500 NLU/L) | |
| 4 | Culture 2 | MB 1 (3.5%) | NA | 200 U/1000 L |
| 5 | Culture 2 | MB 1 (3.5%) | NOLA ™ Fit | 200 U/1000 L |
| | | | (4000 BLU/L) | |
| 6 | Culture 2 | MB 1 (3.5%) | Ha-lactase | 200 U/1000 L |
| | | | (7500 NLU/L) | |
| 7 | Culture 2 | MB 2 (4%) | NA | 200 U/1000 L |
| 8 | Culture 2 | MB 2 (4%) | NOLA ™ Fit | 200 U/1000 L |
| | | | (4000 BLU/L) | |
| 9 | Culture 2 | MB 2 (4%) | Ha-lactase | 200 U/1000 L |
| | | | (7500 NLU/L) | |
| 10 | Culture 2 | MB 3 (4.5%) | NA | 200 U/1000 L |
| 11 | Culture 2 | MB 3 (4.5%) | NOLA ™ Fit | 200 U/1000 L |
| | | | (4000 BLU/L) | |
| 12 | Culture 2 | MB 3 (4.5%) | Ha-lactase | 200 U/1000 L |
| | | | (7500 NLU/L) | |

### Example 1—Fermented Milk with Improved Texture

Gel firmness was measured as complex modulus G* at oscillation frequency of 1.52 Hz as described above. The results presented in the table below shows that addition of either of the two different lactases increase gel firmness measured at day +7 and day +28 irrespective of the protein content of the milk bases and the culture tested.

TABLE 6
Gel firmness measured as complex modulus G* at oscillation frequency
of 1.52 Hz. Measurements were conducted at 13° C. after 7 and 28 days
of storage at 6° C.

| | Complex Modulus G* at | Complex Modulus G* at |
|---|---|---|
| Trial | 1.52 Hz (Pa) Day 7 | 1.52 Hz (Pa) Day 28 |
| 1 | 170 | 177 |
| 2 | 189 | 195 |
| 3 | 176 | 213 |
| 4 | 97 | ND |
| 5 | 103 | ND |
| 6 | 105 | ND |
| 7 | 159 | 177 |
| 8 | 188 | 195 |
| 9 | 189 | 213 |
| 10 | 284 | 309 |
| 11 | 334 | 367 |
| 12 | 302 | 320 |

### Example 2—Fermented Milk with Reduced Post-Acidification

Post-acidification was measured at 6° C. and 25° C. over 7 and 28 days. The results presented in the table below shows that addition of either of two different lactases stabilize and/or reduce post-acidification over shelf life for both cultures tested and the different milk bases, at different protein content.

TABLE 7
Post-acidification measured by pH meter.

| | Post Acidification | Post Acidification | Post Acidification |
|---|---|---|---|
| Trial | at 6° C. | at 6° C. | at 25° C. |
| number | pH Day + 7 | pH Day + 28 | pH Day + 7 |
| 1 | 4.45 | 4.40 | 4.20 |
| 2 | 4.51 | 4.49 | 4.27 |
| 3 | 4.47 | 4.40 | 4.25 |
| 4 | 4.39 | 4.39 | 4.29 |
| 5 | 4.49 | 4.49 | 4.42 |
| 6 | 4.49 | 4.43 | 4.26 |
| 7 | 4.44 | 4.42 | 4.33 |
| 8 | 4.51 | 4.50 | 4.46 |
| 9 | 4.44 | 4.43 | 4.38 |
| 10 | 4.43 | 4.44 | 4.35 |
| 11 | 4.54 | 4.55 | 4.50 |
| 12 | 4.48 | 4.49 | 4.43 |

### Example 3—Fermented Milk with Better Gel Firmness

Milk base with 4.40/protein and low fat (0.11%/) was produced by mixing skimmed milk powder and water in ratio 13:100 during 2 h, followed by batch pasteurization at 80° C. for 20 minutes. Milk was heated to 43° C. and inoculated with different cultures at 0.2 U/L as recommended in the product information sheets provided by the supplier. Milk was incubated at 43° C. until the pH 4.55 was reached. At this point the curd was broken by manual stirring, and the fermented milk was processed using the standard procedure known in the art by pumping through a smoothening valve, heat exchanger where the temperature was reduced to 20° C., and the filling nozzle. Yoghurt samples were collected in standard yoghurt cups and stored at 6° C. for 7 days before rheology analysis.

The rheological properties of the sample were assessed on a rheometer (Anton Paar Modular Compact Rheometer MCR 302, Anton Paar® GmbH, Austria). The rheometer was set to a constant temperature of 13° C. during the time of measurement. The program Stirred_osciIlation+Up_DownFlow was used.

Gel firmness was assessed at 1.52 Hz (Complex modulus G* at 1.52).

The shear stress at 300 1/s was chosen for analysis, as this correlates to texture in mouth (mouthfeel, first impression) and cohesiveness (when swallowing a fermented milk product).

TABLE 8
Gel firmness and Shear Stress of measured in different cultures
of the invention.

| | | Complex | | time to |
|---|---|---|---|---|
| | NOLA Fit | Modulus G* | Shear Stress | pH 4.55 |
| | (BLU/L) | at 1.52 Hz (Pa) | at 300 1/s | (min) |
| Culture 1 | 0 | 202.3 | 54.3 | 508 |
| Culture 1 | 1000 | 240.0 | 53.9 | 644 |
| Culture 1 | 3000 | 223.1 | 63.7 | 740 |
| Culture 2 | 0 | 167.8 | 53.1 | 424 |
| Culture 2 | 1000 | 192.6 | 55.2 | 536 |
| Culture 2 | 3000 | 238.3 | 55.4 | 556 |
| Culture 3 | 0 | 162.5 | 54.0 | 432 |
| Culture 3 | 1000 | 209.3 | 59.0 | 556 |
| Culture 3 | 3000 | 220.0 | 53.6 | 588 |
| Culture 4 | 0 | 163.9 | 52.7 | 436 |
| Culture 4 | 1000 | 246.7 | 52.0 | 484 |
| Culture 4 | 3000 | 203.2 | 53.4 | 500 |

1. A method for producing a fermented milk product comprising-the-steps:
(a) adding a starter culture comprising one or more lactic acid bacteria strains selected from lactic acid bacteria strains deposited at DSMZ-German Collection of Microorganisms and Cell Cultures, Inhoffenstr. 7B, 38124, Braunschweig, Germany (DSMZ) under accession numbers DSM 22935, DSM 22585, DSM 22586, DSM 28910 and DSM 33677, to a milk base;
(b) permitting the starter culture to ferment said milk base until a target pH is reached; and
(c) adding a lactase to the milk base;
wherein the fermented milk product has one or both of improved texture and reduced post-acidification as compared to a fermented milk product produced without addition of the lactase of step (c) or the starter culture of step (a).

2. The method according to claim 1, wherein the starter culture comprises lactic acid bacteria strain DSM 22935, and one or more lactic acid bacteria strains selected from DSM 22585, DSM 22586, DSM 28910, and DSM 33677.

3. The method according to claim 1, wherein the starter culture comprises a combination of lactic acid bacteria strains selected from DSM 22935 and DSM 22586; DSM 22935 and DSM 33677; DSM 22935 and DSM 28910; DSM 22935 and DSM 22585; DSM 22586 and DSM 33677; DSM 22586 and DSM 28910; DSM 22586 and DSM 22585; DSM 33677 and DSM 28910; DSM 33677 and DSM 22585; and DSM 28910 and DSM 22585.

4. The method according to claim 1, wherein the starter culture comprises a combination of lactic acid bacteria strains selected from DSM 22935 and DSM 22586 and DSM 33677; DSM 22935 and DSM 22586 and DSM 28910; DSM 22935 and DSM 22586 and DSM 22585; DSM 22935 and DSM 33677 and DSM 28910; DSM 22935 and DSM 33677 and DSM 22585; and DSM 22935 and DSM 28910 and DSM 22585.

5. The method according to claim 1, wherein the milk base has a protein level of 1-10% by weight.

6. The method according to claim 1, wherein the target pH is in the range of 3.0-6.0.

7. The method according to claim 1, wherein the lactase is added at the start, during, or at the end of the fermentation in step (b).

8. The method according to claim 1, wherein the lactase is selected from a neutral lactase; an acidic lactase; and a low pH stable lactase.

9. The method according to claim 1, wherein texture of the fermented milk product measured at 13° C. as gel firmness by complex modulus in Pa after being stored at a temperature of 6° C. for 7 days after termination of fermentation is increased by at least 5% as compared to a fermented milk product produced without addition of the lactase of step (c) or the starter culture of step (a).

10. The method according to claim 1, wherein pH of the fermented milk product is maintained within a range of 0.30 pH units when stored for 7 days after termination of fermentation at a temperature of 6° C.

11. A fermented milk product obtained by the method according to am claim 1.

12. A fermented milk product with improved texture and/or reduced post-acidification, wherein the product comprises a lactase and starter culture comprising one or more lactic acid bacteria strains selected from DSM 22935, DSM 22585, DSM 22586, DSM 28910 and DSM 33677.

13. A method for improving texture and/or reducing post-acidification of a fermented milk product, comprising preparing a fermented milk product with a starter culture and a lactase selected from a neutral lactase; an acidic lactase; and a low pH stable lactase.

14. The method according to claim 13, wherein the starter culture comprises one or more lactic acid bacteria strains selected from DSM 22935, DSM 22585, DSM 22586, DSM 28910 and DSM 33677.

15. A composition comprising a lactase and two lactic acid bacteria strains selected from DSM 22935 and DSM 22586, DSM 22935 and DSM 33677, DSM 22935 and DSM 28910, DSM 22935 and DSM 22585: DSM 22586 and DSM 33677, DSM 22586 and DSM 28910, DSM 22586 and DSM 22585: DSM 33677 and DSM 28910, DSM 33677 and DSM 22585, and DSM 28910 and DSM 22585.
